(19) United States
(12) Patent Application Publication
(10) Pub. No.: US 2024/0167090 A1
(43) Pub. Date: May 23, 2024

(54) FLAP PROBE AND USE THEREOF

(71) Applicant: TOPPAN INC., Tokyo (JP)

(72) Inventor: Masayuki OGINO, Tokyo (JP)

(73) Assignee: TOPPAN INC., Tokyo (JP)

(21) Appl. No.: 18/498,339

(22) Filed: Oct. 31, 2023

(30) Foreign Application Priority Data
Nov. 2, 2022 (JP) 2022-176209

(51) Int. Cl. C12Q 1/6876; C12N 15/11

(52) U.S. Cl. CPC C12Q 1/6876; C12N 15/11; C12N 2310/315

(57) ABSTRACT

A flap probe for ICA is a single-stranded oligonucleotide having a sequence complementary to a target nucleic acid, and the flap probe has a cleavage resistant structure to the flap endonuclease at least any one position of a position before one nucleotide or a position after one nucleotide of a cleavage position due to a flap endonuclease has cleavage resistance.

### CROSS-REFERENCE TO RELATED APPLICATIONS

This application claims priority under 37 CFR § 119 to Japanese Patent Application No. 2022-176209, filed on Nov. 2, 2022, in the Japan Patent Office, the contents of which are incorporated by reference herein in their entirety.

### REFERENCE TO ELECTRONIC SEQUENCE LISTING

The contents of the electronic sequence listing file in ST.26 XML format (18498339-OSQ21362-UpdateSequenceList.xml); date of creation: Nov. 2, 2022; size of XML file in bytes: 21 KB, is incorporated by reference herein in its entirety.

### BACKGROUND OF THE INVENTION

### Field of the Invention

The present invention relates to a flap probe and use thereof. Specifically, the present invention relates to a method of controlling a cleavage position of a flap probe due to a flap endonuclease in an invasive cleavage assay (ICA), a flap probe used in ICA, a kit for ICA, and a method of detecting a target nucleic acid using ICA.

Priority is claimed on Japanese Patent Application No. 2022-176209, filed on Nov. 2, 2022, the entire content of which is incorporated herein by reference.

### Description of Related Art

As a method of detecting a trace amount of molecules to be analyzed, a method of using fluorescence resonance energy transfer (FRET) is known.

For example, a plurality of methods of accurately and rapidly detecting and quantifying target nucleic acids are present in gene diagnosis. Among these, the invasive cleavage assay (ICA) has excellent operability and reaction stability (see, for example, Eis P. S. et al., “An invasive cleavage assay for direct quantitation of specific RNAs.”, Nature Biotechnology, Vol. 19, Issue 7, pp. 673-676, 2001).

Here, ICA will be described with reference to FIG. 5. FIG. 5 is a schematic view showing an example of ICA in the related art. In the example of FIG. 5, the presence of a thymine (T) base 101 in a target nucleic acid 100 is detected. First, a flap probe 210 and an invasive probe 120 that are complementary to the target nucleic acid 100 hybridize. As a result, the invasive probe 120 hybridizes with a site of the target nucleic acid 100, which is adjacent to a position where the flap probe 210 hybridizes. Further, at least one nucleotide at the 3′ end of the invasive probe 120 enters a position of the 5′ end of a region 241 where the flap probe 210 and the target nucleic acid 100 hybridize to form a first triple-stranded structure 230.

Subsequently, in a case where a flap endonuclease reacts with the first triple-stranded structure 230, a flap site 240 of the first triple-stranded structure 230 is cleaved, and a nucleic acid fragment 240 is generated. Subsequently, the nucleic acid fragment 240 hybridizes with a fluorescent probe 150 to form a second triple-stranded structure 260.

In the example of FIG. 5, a fluorescent substance F is bound to the 5′ end of the fluorescent probe 150, and a quenching substance Q is bound to the 3′ side of several nucleotides from the 5′ end of the fluorescent probe 150. The fluorescent substance F and the quenching substance Q are positioned spatially in the vicinity. Therefore, the fluorescence emitted by the fluorescent substance F is quenched by the quenching substance Q.

Subsequently, in a case where a flap endonuclease reacts with the second triple-stranded structure 260, a flap site 170 of the second triple-stranded structure 260 is cleaved, and a nucleic acid fragment 170 is generated. As a result, the fluorescent substance F is released from the quenching substance Q, and fluorescence is emitted by irradiation with excitation light. The presence of the thymine (T) base 101 in the target nucleic acid 100 can be detected by detecting the fluorescence.

### SUMMARY OF THE INVENTION

However, the present inventors found that in ICA, a product cleaved at a main cleavage position, that is the nucleic acid fragment 240, a by-product cleaved at one nucleotide before the main cleavage position 240a, and a by-product cleaved at one nucleotide after the main cleavage position 240b are present as shown in FIG. 5. Due to the presence of such by-products, an appropriate second triple-stranded structure 260 cannot be formed, and the reaction efficiency of ICA is reduced.

The present invention has been made in consideration of the above-described circumstances, and an object thereof is to provide a flap probe for ICA in which a cleavage position due to a flap endonuclease is controlled, a method of controlling a cleavage position of the flap probe due to a flap endonuclease in ICA using the flap probe, a kit for ICA, and a method of detecting a target nucleic acid using ICA.

That is, the present invention includes the following aspects.

(1) A method of controlling a cleavage position in an invasive cleavage assay, the method includes bringing a target nucleic acid, a flap probe, and an invasive probe into contact with each other, in which the flap probe is a single-stranded oligonucleotide having a sequence complementary to the target nucleic acid; and the flap probe has a structure for cleavage resistance to the flap endonuclease at least any one position of a position before one nucleotide or a position after one nucleotide of the cleavage position due to the flap endonuclease.

(2) The method according to (1), in which the structure for cleavage resistance to the flap endonuclease is a phosphothioate bond.

(3) A flap probe which is used in an invasive cleavage assay, in which the flap probe is a first single-stranded oligonucleotide having a sequence complementary to a first position of a target nucleic acid; and the flap probe has a structure for cleavage resistance to the flap endonuclease at least any one position of a position before one nucleotide or a position after one nucleotide of a cleavage position due to a flap endonuclease.

(4) The flap probe according to (3), in which the structure for cleavage resistance to the flap endonuclease is a phosphothioate bond.

(5) A kit for an invasive cleavage assay, includes the flap probe according to (3) or (4); an invasive probe; a fluorescent probe; and a flap endonuclease, in which the invasive probe is a second single-stranded oligonucleotide having a second sequence complementary to a second position of the target nucleic acid; the first and second sequences are different from each other; and the first and second positions are located adjacent to each other in the target nucleic acid; the fluorescent probe is a third single-stranded oligonucleotide labeled with a fluorescent substance and a quenching substance; and a 5′side of the fluorescent probe has a hairpin structure formed by self-hybridization; a 3′ side of the fluorescent probe has a sequence complementary to a nucleic acid fragment included in the flap probe.

(5′) The kit according to (5), in which a triple-stranded structure is formed at a 5′ end portion by hybridization of the nucleic acid fragment; cleavage is made by the flap endonuclease that recognizes the triple-stranded structure; the fluorescent substance is released from the quenching substance; and fluorescence is emitted by irradiation with excitation light.

(6) A method of detecting a target nucleic acid using an invasive cleavage assay, the method includes using the flap probe according to (3) or (4).

According to the flap probe of the above-described aspect, it is possible to provide a flap probe for ICA in which a cleavage position due to a flap endonuclease is controlled. According to the method of the above-described aspect, it is possible to control a cleavage position of the flap probe due to the flap endonuclease in ICA. The kit for ICA according to the above-described aspect includes the flap probe and can control a cleavage position of the flap probe due to the flap endonuclease in ICA. The method of detecting a target nucleic acid using ICA according to the above-described aspect uses the flap probe, and the cleavage position of the flap probe due to the flap endonuclease can be controlled in ICA.

### BRIEF DESCRIPTION OF THE DRAWINGS

FIG. 1 is a schematic view showing an example of an invasive cleavage assay (ICA).

FIG. 2 is a graph showing results of Example 1.

FIG. 3 is a graph showing results of Example 1.

FIG. 4 is a graph showing results of Example 2.

FIG. 5 is a schematic view showing an example of an ICA in the related art.

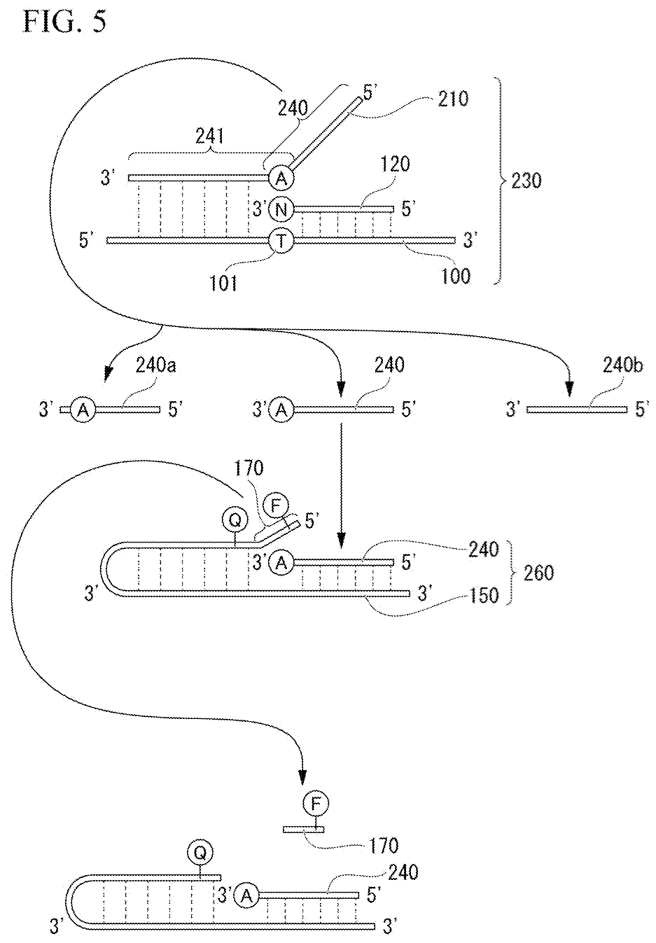

### DETAILED DESCRIPTION OF THE INVENTION

<<Flap Probe for ICA>>

The flap probe for ICA of the present embodiment is formed of a single-stranded oligonucleotide having a sequence complementary to a target nucleic acid, and the flap probe has a structure for cleavage resistance the flap endonuclease at least any one position of a position before one nucleotide or a position after one nucleotide of a cleavage position due to a flap endonuclease.

In the present specification, “a position before one nucleotide of a cleavage position” refers to a position one nucleotide closer to a 3′ end than the cleavage position. “A position after one nucleotide of a cleavage position” refers to a position one nucleotide closer to a 5′ end than the cleavage position.

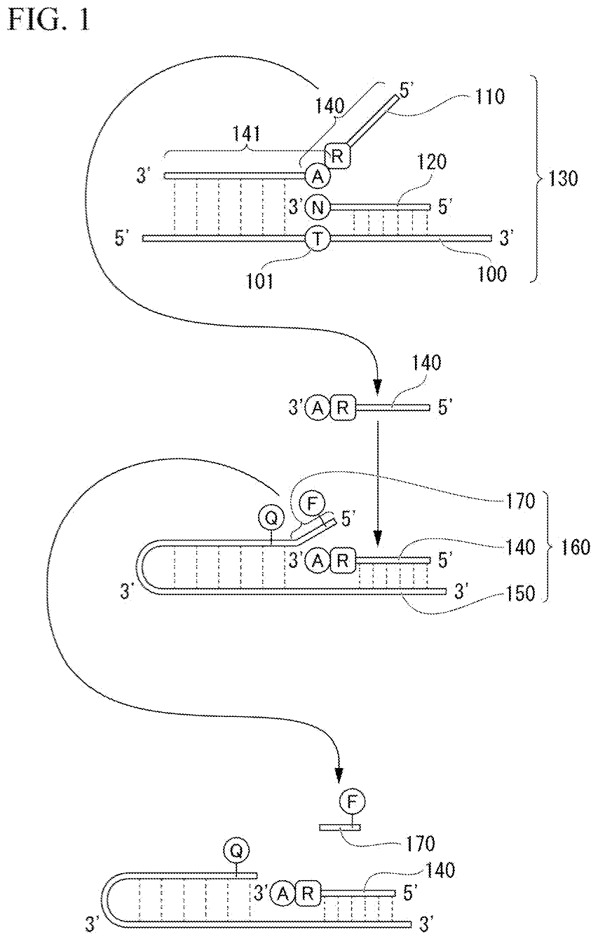

Since the flap probe for ICA according to the present embodiment has the above-described structure, only a product cleaved at a main cleavage position can be preferentially generated and generation of a by-product cleaved at one nucleotide before and after the main cleavage position can be suppressed, by controlling the cleavage position due to the flap endonuclease in ICA. In this manner, since a nucleic acid fragment 140 suitable for forming a second triple-stranded structure 160 shown in FIG. 1 which will be described in detail later is generated, the reactivity of ICA can be improved, and the reaction speed can be increased.

That is, one embodiment of the present invention provides a method of controlling a cleavage position in ICA, the method includes bringing a target nucleic acid, a flap probe, and an invasive probe into contact with each other, in which the flap probe is a single-stranded oligonucleotide having a sequence complementary to the target nucleic acid, and the flap probe has a structure for cleavage resistance to the flap endonuclease at least any one position of a position before one nucleotide or a position after one nucleotide of a cleavage position due to a flap endonuclease.

The method according to the embodiment described above is a method of controlling a cleavage position of a flap probe due to a flap endonuclease in ICA, including preparing a target nucleic acid, a flap probe, and an invasive probe, bringing the target nucleic acid, the flap probe, and the invasive probe into contact with each other, and bringing a flap endonuclease into contact with a reactant by the contact, in which the flap probe is a single-stranded oligonucleotide having a sequence complementary to a target nucleic acid, and the flap probe has a structure for cleavage resistance to the flap endonuclease at least any one position of a position before one nucleotide or a position after one nucleotide of a cleavage position due to a flap endonuclease, the invasive probe is a single-stranded oligonucleotide having a sequence complementary to a target nucleic acid, the sequence complementary to the target nucleic acid of the flap probe and the sequence complementary to the target nucleic acid of the invasive probe are different from each other, the flap probe and the invasive probe hybridize with positions adjacent to each other in the target nucleic acid to form a triple-stranded structure, the flap endonuclease recognizes the triple-stranded structure, and the flap probe is cleaved at a cleavage position due to the flap endonuclease so that a nucleic acid fragment is cleaved.

Further, the method according to the embodiment described above can also be considered as a method of improving reactivity of ICA.

In the present specification, the improvement of the reactivity of ICA may denote that a fluorescence signal detected as a result of ICA reaches a predetermined value in a shorter time. Alternatively, the fluorescence signal intensity detected as a result of ICA may reach a higher value. Alternatively, a background fluorescence signal detected as a result of ICA may be maintained at a lower value.

It can be said that the flap probe for ICA according to the present embodiment is a substrate of a flap endonuclease since the flap probe is cleaved by the flap endonuclease.

Examples of the flap endonuclease include flap endonuclease 1 (NCBI accession number: WP_011012561.1, Holliday junction 5′ flap endonuclease (GENI) (NCBI accession number: NP_001123481.3) and excision repair protein (NCBI accession number: AAC37533.1).

As shown in FIG. 1, the flap probe 210 includes a nucleic acid fragment 140, a region which hybridizes with a site of the target nucleic acid 100, and a structure for cleavage resistance to the flap endonuclease R. In the flap probe 210 according to the present embodiment, the structure for cleavage resistance to the flap endonuclease R may include a nucleic acid at a position before one nucleotide or a nucleic acid at a position after one nucleotide of the cleavage position due to the flap endonuclease or may include both a nucleic acid at a position before one nucleotide and a nucleic acid at a position after one nucleotide of the cleavage position due to the flap endonuclease. As described in the examples below, in a case where at least any one nucleic acid at a position before one nucleotide or at a position after one nucleotide of the cleavage position due to the flap endonuclease has the structure for cleavage resistance to the flap endonuclease R, the time at which the S/N (Signal/Noise) ratio is maximized is shorter than that of a flap probe that does not have the structure for cleavage resistance to the flap endonuclease R. FIG. 1 illustrates the structure for cleavage resistance to the flap endonuclease R at a position after one nucleotide of the cleavage position due to the flap endonuclease as one example.

Further, nucleic acids at positions before and after two, three, four, or five nucleotides of the cleavage position due to the flap endonuclease in addition to at least any one nucleic acid at a position before one nucleotide or a position after one nucleotide of the cleavage position due to the flap endonuclease may further have the structure for cleavage resistance to the flap endonuclease R.

It is preferable that the structure for cleavage resistance to the flap endonuclease R is a phosphothioate bond. In a case where a phosphodiester bond is substituted with a phosphothioate bond in at least any one nucleic acid at a position before one nucleotide or at a position after one nucleotide of the cleavage position due to the flap endonuclease, the cleavage resistance to the flap endonuclease can be imparted to the flap probe.

Further, the substitution of the phosphodiester bond of the nucleic acid at the position before one nucleotide of the cleavage position due to the flap endonuclease with a phosphothioate bond denotes that the phosphodiester bond between the nucleic acid at the position before one nucleotide of the cleavage position due to the flap endonuclease and the nucleic acid of the cleavage position due to the flap endonuclease is substituted with a phosphothioate bond. Further, the substitution of the phosphodiester bond of the nucleic acid at the position after one nucleotide of the cleavage position due to the flap endonuclease with a phosphothioate bond denotes that the phosphodiester bond between the nucleic acid at the position after one nucleotide of the cleavage position due to the flap endonuclease and the nucleic acid after two nucleotides of the cleavage position due to the flap endonuclease is substituted with a phosphothioate bond. As described above, the phosphothioate bond between the nucleic acid at the position before one nucleotide of the cleavage position due to the flap endonuclease and the nucleic acid of the cleavage position due to the flap endonuclease is defined to be included by the nucleic acid at the position before one nucleotide of the cleavage position due to the flap endonuclease in the specification of the present application. The phosphothioate bond between the nucleic acid at the position after one nucleotide of the cleavage position due to the flap endonuclease and the nucleic acid after two nucleotides of the cleavage position due to the flap endonuclease is defined to be included by the nucleic acid at the position after one nucleotide of the cleavage position due to the flap endonuclease in the specification of the present application.

Further, the cleavage resistance to the flap endonuclease may be imparted to the flap probe by substituting at least any one nucleic acid at a position before one nucleotide or at a position after one nucleotide of the cleavage position due to the flap endonuclease with a bridged nucleic acid (BNA) or a locked nucleic acid (LNA).

It is preferable that the single-stranded oligonucleotide forming the flap probe 110 according to the present embodiment has a length of approximately 15 nucleotides or greater and 30 nucleotides or less. Further, it is preferable that the sequence complementary to the target nucleic acid 100 has a length of 8 nucleotides or greater and nucleotides or less.

In the single-stranded oligonucleotide forming the flap probe 110 according to the present embodiment, the flap site cleaved by the flap endonuclease corresponds to the nucleic acid fragment 140 shown in FIG. 1. Since the nucleic acid fragment 140 hybridizes with the fluorescent probe 150 to form the second triple-stranded structure 160, it is preferable that the single-stranded oligonucleotide forming the flap probe 110 according to the present embodiment has a sequence complementary to the 3′ side of the fluorescent probe 150. It is preferable that the sequence complementary to the 3′ side of the fluorescent probe 150 has a length of approximately 8 nucleotides or greater and 15 nucleotides or less.

<Oligonucleotide>

The single-stranded oligonucleotide forming the flap probe 110 according to the present embodiment is not particularly limited as long as the effects of the present invention are exhibited, and the single-stranded oligonucleotide may be a natural nucleic acid or a synthesized nucleic acid.

Examples of the natural nucleic acid include genomic DNA, mRNA, rRNA, hnRNA, miRNA, tRNA, and the like. The natural nucleic acids may be recovered from a living body or recovered from water, organic substances, and the like brought into contact with a living body. Examples of a method of recovering natural nucleic acids include known methods such as a phenol/chloroform method.

Examples of the synthesized nucleic acid include synthetic DNA, synthetic RNA, cDNA, and the like.

A method of synthesizing the oligonucleotide is not particularly limited, and examples thereof include known chemical synthesis methods such as DNA solid phase synthesis by a phosphoramidite method.

The flap probe 110 according to the present embodiment can be synthesized by the above-described synthesis method using a nucleic acid that has a structure for cleavage resistance to the flap endonuclease, such as a nucleic acid in which a phosphodiester bond is substituted with a phosphothioate bond, and a nucleic acid that does not have a structure for cleavage resistance to a flap endonuclease.

The nucleic acid in which a phosphodiester bond is substituted with a phosphothioate bond can be synthesized by, for example, the method described in Lyer R P et al., “3H-1,2-Benzodithiole-3-one 1,1-Dioxide as an Improved Sulfurizing Reagent in the Solid-Phase Synthesis of Oligodeoxyribonucloside Phoshorothioates.”, J. Am. Chem. Soc. Vol. 112, pp. 1253-1254, 1990. or the like.

Specifically, the nucleic acid can be synthesized by reacting 3H-1,2-benzodithiole-3-one 1,1-dioxide (1) with a dinucleotide (2), as represented by Reaction Formula (A). In Reaction Formula (A), Base denotes a base, which is an optional base such as adenine, cytosine, guanine, thymine, or uracil.

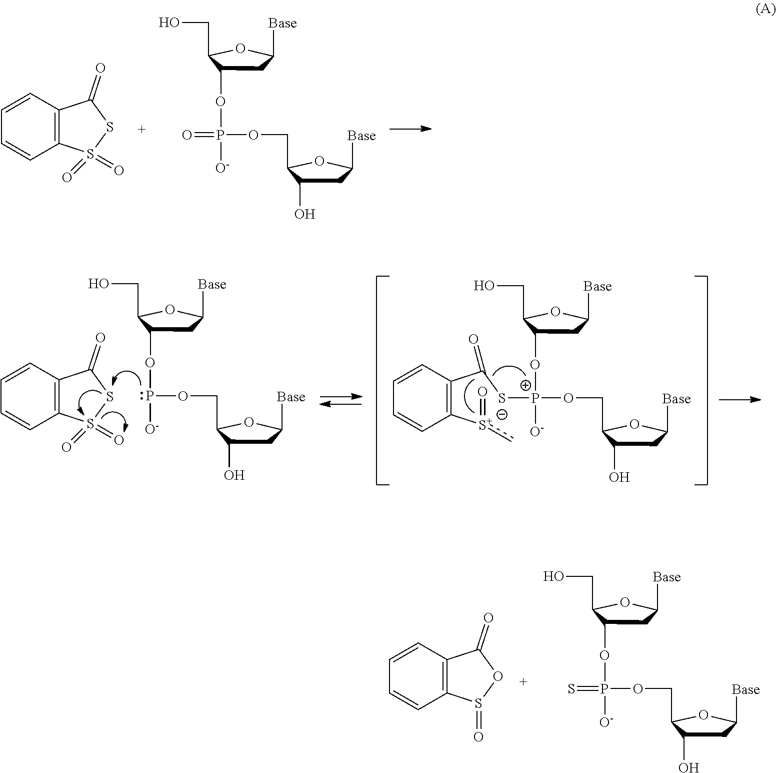

<<Kit for ICA>>

A kit for ICA according to the present embodiment includes the flap probe described above, an invasive probe, a fluorescent probe, and a flap endonuclease.

In the kit for ICA according to the present embodiment, those described in the section “flap probe” can be used as the flap probe 110 and the flap endonuclease.

Since the kit for ICA according to the present embodiment has the above-described flap probe 110, only a product cleaved at a main cleavage position can be preferentially generated and generation of a by-product cleaved at one nucleotide before and after the main cleavage position can be suppressed, by controlling the cleavage position due to the flap endonuclease of the flap probe in ICA. As a result, since the nucleic acid fragment 140 suitable for forming the second triple-stranded structure 160 shown in FIG. 1 is generated, the reactivity of ICA can be improved, and the reaction speed can be increased.

<Invasive Probe>

An invasive probe 120 is a single-stranded oligonucleotide including a sequence complementary to a target nucleic acid 100. It is preferable that the invasive probe 120 is a single-stranded oligonucleotide formed only of a sequence complementary to the target nucleic acid 100.

The sequence complementary to the target nucleic acid of the flap probe 110 and the sequence complementary to the target nucleic acid of the invasive probe 120 are sequences different from each other. As shown in FIG. 1, the flap probe 110 and the invasive probe 120 hybridize with positions adjacent to each other in the target nucleic acid 100.

It is preferable that the single-stranded oligonucleotide forming the invasive probe 120 has a length of approximately 15 nucleotides or greater and 30 nucleotides or less. Further, it is preferable that the sequence complementary to the target nucleic acid 100 has a length of 15 nucleotides or greater and 30 nucleotides or less.

<Fluorescent Probe>

The fluorescent probe 150 is a single-stranded oligonucleotide labeled with a fluorescent substance F and a quenching substance Q.

The 5′ side of the fluorescent probe 150 has a hairpin structure formed by self-hybridization. The 3′ side of the fluorescent probe 150 has a sequence complementary to a nucleic acid fragment 140 formed by cleavage of the flap probe due to a flap endonuclease. A triple-stranded structure 160 is formed at a 5′ end portion of the fluorescent probe 150 by hybridization of the nucleic acid fragment 140. The fluorescent probe 150 is cleaved by the flap endonuclease that recognizes the triple-stranded structure 160 to release the fluorescent substance F from the quenching substance Q, and fluorescence is emitted by irradiation with excitation light.

Here, a triple-stranded structure 160 formed by a formation of a hairpin structure at the 5′ side of the fluorescent probe 150 formed by self-hybridization and a hybridization of the nucleic acid fragment 140 formed by cleavage of the flap probe due to the flap endonuclease with the 3′ side of the fluorescent probe 150 corresponds to the second triple-stranded structure 160 shown in FIG. 1. That is, the nucleic acid fragment 140 that hybridizes with the 3′ side of the fluorescent probe 150 and is formed by cleavage due to the flap endonuclease corresponds to the flap site 140 derived from the flap probe 110.

It is preferable that the fluorescent probe 150 has a length of approximately 20 nucleotides or greater and 150 nucleotides or less. Further, the number of nucleotide pairs forming the hairpin structure is preferably approximately 5 or greater and 50 or less.

(Fluorescent Substance)

The fluorescent substance F is not particularly limited, and examples thereof include fluorescein (molecular weight of 332.3), ATT0425 (molecular weight of 401.45), Alexa488 (molecular weight of 534.47), ATT0542 (molecular weight of 914), Yakima Y (molecular weight of 718.33), Redmond R (molecular weight of 445.3), ATT0643 (molecular weight of 836), Alexa647 (molecular weight of 860), Alexa680 (molecular weight of 1050), Alexa568 (molecular weight of 694.7), FAM (molecular weight of 332.3), ATT0633 (molecular weight of 652.2), Cy5 (molecular weight of 483.7), HiLyte Fluor 647 (molecular weight of 1205.6), ATT0665 (molecular weight of 723), Alexa594 (molecular weight of 722.8), Cy3 (molecular weight of 457.6), and ROX (molecular weight of 534.6). The molecular weight of the fluorescent substance F is preferably 350 or greater and 1100 or less, more preferably 445 or greater and 1100 or less, and still more preferably 445 or greater and 914 or less. In a case where the molecular weight of the fluorescent substance F is in the above-described ranges, a higher signal-to-noise ratio (S/N ratio) tends to be obtained.

(Quenching Substance)

The quenching substance Q is not particularly limited as long as the quenching substance can quench the fluorescence of the fluorescent substance F to be used, and Examples thereof include Black Hole Quencher (BHQ) (registered trademark)-1, BHQ (registered trademark)-2, BHQ (registered trademark)-3, Tide Quencher 1 (TQ1), Tide Quencher 2 (TQ2), Tide Quencher 2WS (TQ2WS), Tide Quencher 3 (TQ3), Tide Quencher 3WS (TQ3WS), Tide Quencher 4 (TQ4), Tide Quencher 4WS (TQ4WS), Tide Quencher 5 (TQ5), Tide Quencher 5WS (TQSWS), Tide Quencher 6WS (TQ6WS), Tide Quencher 7WS (TQ7WS), QSY35, QSY7, QSY9, QSY21, Iowa Black FQ, and Iowa Black RQ. As the quenching substance Q, a quenching substance Q capable of quenching the fluorescence of the fluorescent substance F to be used is selected.

In a case where the fluorescent substance F is in the spatial vicinity of the quenching substance Q, the fluorescence from the fluorescent substance F is quenched by the quenching substance Q. Here, “fluorescence is quenched” denotes as follows. In a case where the quenching substance Q is not present, the intensity of fluorescence emitted from the fluorescent substance F in a case where the fluorescent substance F is irradiated with excitation light is defined as A. Further, in a case where the quenching substance Q is present in the spatial vicinity of the fluorescent substance F, the intensity of fluorescence emitted from the fluorescent substance F in a case where the fluorescent substance F is irradiated with the excitation light is defined as B. Here, “fluorescence is quenched” denotes that the value of B/A is 0.4 or less.

The distance between the fluorescent substance F and the quenching substance Q in a state where the fluorescent substance F is in the spatial vicinity of the quenching substance Q is not particularly limited as long as the emission of fluorescence from the fluorescent substance F is suppressed by the quenching substance Q. The distance between the fluorescent substance F and the quenching substance Q is preferably 10 nm or less, more preferably 5 nm or less, and still more preferably 2 nm or less.

The single-stranded oligonucleotide forming the invasive probe 120 and the fluorescent probe 150 is not particularly limited as long as the effects of the present invention are exhibited, and the single-stranded oligonucleotide may be a natural nucleic acid or a synthetic nucleic acid. Examples of the kind of nucleic acid include the same nucleic acids as described in the section of “flap probe”.

<<Method of Detecting Target Nucleic Acid Using ICA>>

A method of detecting a target nucleic acid 100 using ICA according to the present embodiment includes using the flap probe 110 described above.

Since the method of detecting a target nucleic acid 100 using ICA according to the present embodiment includes using the flap probe 110 described above, only a product cleaved at a main cleavage position can be preferentially generated and generation of a by-product cleaved at one nucleotide before and after the main cleavage position can be suppressed, by controlling the cleavage position due to the flap endonuclease of the flap probe in ICA. As a result, since the nucleic acid fragment 140 suitable for forming the second triple-stranded structure 160 shown in FIG. 1 is generated, the reactivity of ICA can be improved, and the reaction speed can be increased.

In the method of detecting a target nucleic acid using ICA according to the present embodiment, it is preferable to use an invasive probe 120, a fluorescent probe 150, and a flap endonuclease in addition to the flap probe 110 described above. That is, in one embodiment, the present invention provides a method of detecting a target nucleic acid 100 using ICA, including applying the above-described kit for ICA.

The method according to the present embodiment is a method of detecting a target nucleic acid using ICA, the method including preparing a flap probe, an invasive probe, and a fluorescent probe, bringing the target nucleic acid, the flap probe, and the invasive probe into contact with each other, bringing a flap endonuclease into contact with a reactant obtained by bringing the target nucleic acid, the flap probe, and the invasive probe into contact with each other to generate a first nucleic acid fragment cleaved by the flap endonuclease, bringing the first nucleic acid fragment and the fluorescent probe into contact with each other, and bringing a flap endonuclease into contact with a reactant obtained by bringing the first nucleic acid fragment and the fluorescent probe into contact with each other to generate a second nucleic acid fragment cleaved by the flap endonuclease, in which the flap probe is a single-stranded oligonucleotide having a sequence complementary to the target nucleic acid, and the flap probe has a structure for cleavage resistance to the flap endonuclease at least any one position of a position before one nucleotide or a position after one nucleotide of a cleavage position due to a flap endonuclease, the invasive probe is a single-stranded oligonucleotide having a sequence complementary to the target nucleic acid, the sequence complementary to the target nucleic acid of the flap probe and the sequence complementary to the target nucleic acid of the invasive probe are different from each other, the flap probe and the invasive probe hybridize with positions adjacent to each other in the target nucleic acid to form the triple-stranded structure, the flap endonuclease recognizes the triple-stranded structure, and the flap probe is cleaved at a cleavage position due to the flap endonuclease so that a first nucleic acid fragment is cleaved out, the fluorescent probe is a single-stranded oligonucleotide labeled with a fluorescent substance and a quenching substance, the 5′ side of the fluorescent probe has a hairpin structure formed by self-hybridization, the 3′ side of the fluorescent probe has a sequence complementary to the first nucleic acid fragment formed by cleavage of the flap probe due to the flap endonuclease, a triple-stranded structure is formed at a 5′ end portion by hybridization of the first nucleic acid fragment, cleavage is made by the flap endonuclease that recognizes the triple-stranded structure, the fluorescent substance is released from the quenching substance, and fluorescence is emitted by irradiation with excitation light.

Here, a method of detecting a target nucleic acid using ICA of the present embodiment will be described with reference to FIG. 1. FIG. 1 is a schematic view showing an example of method of detecting a target nucleic acid using ICA of the present embodiment. In the example of FIG. 1, the presence of a thymine (T) base 101 in a target nucleic acid 100 is detected. The flap probe 110 includes the structure for cleavage resistance to the flap endonuclease R. First, a flap probe 110 and an invasive probe 120 that are complementary to the target nucleic acid 100 hybridize. As a result, the invasive probe 120 hybridizes with a site of the target nucleic acid 100, which is adjacent to a position where the flap probe 110 hybridizes with the target nucleic acid 100. Further, at least one nucleotide at the 3′ end of the invasive probe 120 enters a position of the 5′ end of a region 141 where the flap probe 110 and the target nucleic acid 100 hybridize to form a first triple-stranded structure 130.

Subsequently, in a case where a flap endonuclease reacts with the first triple-stranded structure 130, a flap site 140 of the first triple-stranded structure 130 is cleaved, and a nucleic acid fragment 140 is generated. Subsequently, the nucleic acid fragment 140 hybridizes with a fluorescent probe 150 to form a second triple-stranded structure 160.

In the example of FIG. 1, a fluorescent substance F is bound to the 5′ end of the fluorescent probe 150, and a quenching substance Q is bound to the 3′ side of several nucleotides from the 5′ end of the fluorescent probe 150. The fluorescent substance F and the quenching substance Q are positioned spatially in the vicinity. Therefore, the fluorescence emitted by the fluorescent substance F is quenched by the quenching substance Q.

Subsequently, in a case where a flap endonuclease reacts with the second triple-stranded structure 160, a flap site 170 of the second triple-stranded structure 160 is cleaved, and a nucleic acid fragment 170 is generated. As a result, the fluorescent substance F is released from the quenching substance Q, and fluorescence is emitted by irradiation with excitation light. The presence of the thymine (T) base 101 in the target nucleic acid 100 can be detected by detecting the fluorescence.

In the method according to the present embodiment, it is preferable that ICA is performed in a microspace. Specifically, it is preferable to perform ICA using a device having a well array with a plurality of minute wells.

The well may be used as it is without being treated, and may be subjected to a pretreatment of immobilizing an extraction reagent or a detection reagent such as an antibody on an inner wall of the well in advance or covering an opening portion of the well with a lipid bilayer depending on the purpose thereof.

The device may have a flow path, and a reaction solution of ICA may be supplied to the well of the well array through the flow path.

The diameter of the well may be, for example, approximately 3 μm, and the depth of the well may be, for example, approximately 4.5 The wells may be arranged on a base material such that a triangular lattice shape or a square lattice shape is formed, to form a well array.

Examples of the material of the base material include a cycloolefin polymer, a cycloolefin copolymer, silicone, polypropylene, polycarbonate, polystyrene, polyethylene, polyvinyl acetate, a fluororesin, and an amorphous fluororesin.

The number of wells in the well array is preferably approximately in a range of 100000 to 6000000 per device. Further, the volume per well is preferably in a range of 1 fL to 6 pL.

### EXAMPLES

Hereinafter, the present invention will be described based on the examples, but the present invention is not limited to the following examples.

### Example 1

(Examination on Flap Probe)

Phosphodiester bonds of nucleotides in the vicinity of a cleavage position of a flap probe due to a flap endonuclease were substituted with a phosphothioate bond to impart cleavage resistance to the flap endonuclease. ICA was performed using the flap probe, and the reactivity was compared.

ICA was performed using the nucleic acid fragment listed in Table 1 below. The target nucleic acid (SEQ ID NO: 1) in Table 1 is a nucleic acid as a target to be detected. In ICA of the present experimental example, the presence of guanine (g) indicated by a small letter in the target nucleic acid was detected. The flap probes (SEQ ID NOS: 2 to 6) and the invasive probe (SEQ ID NO: 7) each have a base sequence complementary to the target nucleic acid (SEQ ID NO: 1).

In a case where the flap probes (SEQ ID NOS: 2 to 6) and the invasive probe (SEQ ID NO: 8) hybridize to the target nucleic acid (SEQ ID NO: 1), a triple-stranded structure is formed. Here, “5′-CGCGCCGAGGC-3′” (SEQ ID NO: 9) of the flap probes (SEQ ID NOS: 2 to 6) does not form base pairs but forms a flap site.

The flap endonuclease recognizes the above-described triple-stranded structure and cleaves the flap probes (SEQ ID NOS: 2 to 6), and the nucleic acid fragment 1 (SEQ ID NO: 9) is typically cleaved out. Here, in the flap probe standard (SEQ ID NO: 2), a nucleic acid fragment 2 (SEQ ID NO: 10) that is one nucleotide shorter than a nucleic acid fragment 1 (SEQ ID NO: 9) and a nucleic acid fragment 3 (SEQ ID NO: 11) that is one nucleotide longer than the nucleic acid fragment 1 (SEQ ID NO: 9) are cleaved out as a by-product.

TABLE 1

| | SEQ | |
|---|---|---|
| | ID | |
| Name | NO: | Base sequence (5′ → 3′) |
| Target | 1 | CCGAAGGGCATGAGCTGCgTGATGAGCTGCACGGT |
| nucleic | | GGAGGTGAGGCAGATGCCCAGCAGGCGGCACACGT |
| acid | | GGGGGTTGTCCACGCTGGCCATCACGTAGGCTTCC |
| Flap probe | 2 | CGCGCCGAGGpCpGpCAGCTCATGCCC |
| standard | | |
| Flap probe | 3 | CGCGCCGAGGsCpGpCAGCTCATGCCC |
| 10S | | |
| Flap probe | 4 | CGCGCCGAGGpCsGpCAGCTCATGCCC |
| 11S | | |
| Flap probe | 5 | CGCGCCGAGGpCpGsCAGCTCATGCCC |
| 12S | | |
| Flap probe | 6 | CGCGCCGAGGsCpGsCAGCTCATGCCC |
| 10S12S | | |
| Flap probe | 7 | CGCGCCGAGGpCpGpCAGCTCATGCsCsC |
| 22S23S | | |
| Invasive | 8 | CCACCGTGCAGCTCATCAA |
| probe | | |
| Nucleic acid | 9 | CGCGCCGAGGC |
| fragment 1 | | |
| Nucleic acid | 10 | CGCGCCGAGG |
| fragment 2 | | |
| Nucleic acid | 11 | CGCGCCGAGGCG |
| fragment 3 | | |
| Fluorescent | 12 | F-tCT-T(Q)-AGCCGGTTTTCCGGCTGAGACCTC |
| probe | | GGCGCG |

p written in a small letter in the flap probes (SEQ ID NOS: 2 to 6) listed in Table 1 denotes a phosphodiester bond in a sugar phosphate skeleton, and s in a small letter in the flap probes 10S, 11S, 12S, 10S12S, and 22S23S (SEQ ID NOS: 3 to 6) denotes a phosphothioate bond. The fluorescent probe (SEQ ID NO: 12) is a fluorescent probe in which a fluorescent substance is bonded to a base. F in the fluorescent probe represents Alexa Fluor (registered trademark) 488, and Q represents BHQ (registered trademark)-1.

In a case where the cleaved-out nucleic acid fragment 1 (SEQ ID NO: 9) hybridizes with the fluorescent probe, the thymine (t) residue written in a small letter in Table 1 of the fluorescent probe forms a triple-stranded structure, serves as a substrate of the flap endonuclease, and is cleaved. As a result, the fluorescent substance is separated from the quenching substance, and a fluorescent signal is detected by irradiation with excitation light.

First, each reaction solution having the composition listed in Table 2 below was prepared and poured into a micro-test tube. Subsequently, a change in fluorescence intensity (excitation wavelength: 490 nm, fluorescence wavelength: 520 nm) in a case where the tube was set in a real-time PCR device and heated at 66° C. for 60 minutes was measured over time. For comparison, a reaction solution in which sterilized water was added instead of the target nucleic acid was prepared, and the same measurement as described above was performed.

TABLE 2

| Reagent | Final concentration | |
|---|---|---|
| Target nucleic acid | 1 | μM |
| Flap probe | 1 | μM |
| Invasive probe | 1 | μM |
| Fluorescent probe | 4 | μM |
| Tris (pH 8.5) | 25 | mM |
| NaCl | 20 | mM |
| MgCl₂ | 25 | mM |
| Tween 20 | 0.05 | (v/v)% |
| Flap endonuclease | 1,000 | U |
| Distilled water | Appropriate amount | |
| Total amount | 10 | μL |

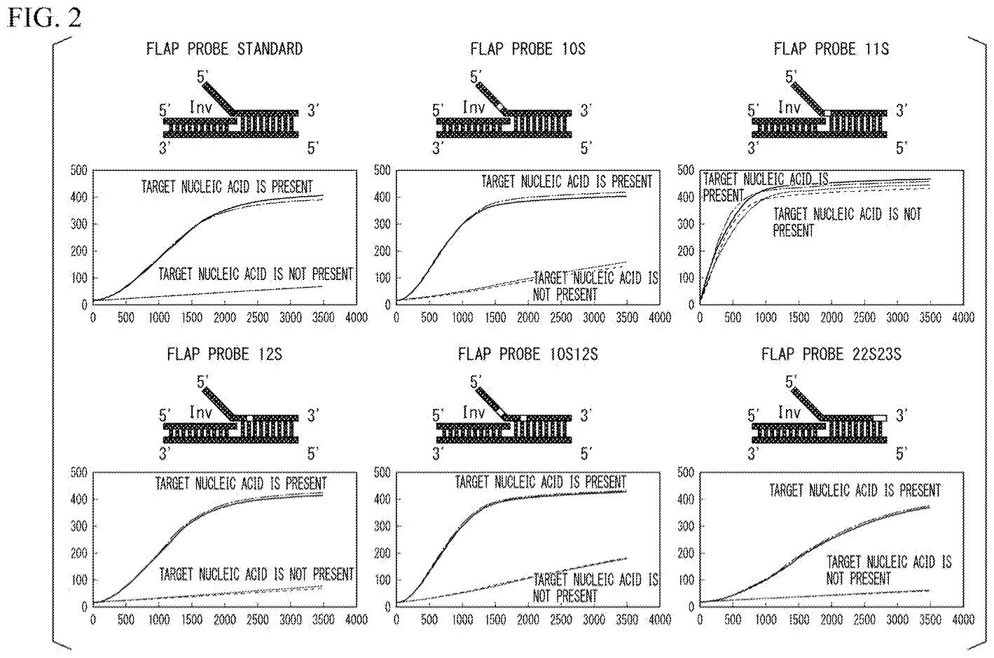

FIG. 2 is a graph showing results of measuring changes in fluorescence intensity in a case where each flap probe was used. In FIG. 2, the horizontal axis represents the elapsed time (seconds) from the start of the reaction, and the vertical axis represents the fluorescence intensity (relative value). For each sample, measurement was performed at N=2.

As shown in FIG. 2, it was clarified that the position before and after one nucleotide of the 11th nucleic acid from the 5′ end, which is a cleavage site in the flap probe, that is, the phosphodiester bond in the sugar phosphate skeleton of at least any one nucleic acid of the 10th nucleic acid from the 5′ end or the 12th nucleic acid from the 5′ end was substituted with a phosphothioate bond, the rise of fluorescence intensity was faster and the reactivity of ICA was improved. Further, it was found that the fluorescence intensity was likely to rise faster in a case where the phosphodiester bond in the sugar phosphate skeleton of the 10th nucleic acid from the 5′ end was substituted with a phosphothioate bond. The fluorescence intensity under a condition that no target nucleic acid was present, that is background, was suppressed in a case where the phosphodiester bond in the sugar phosphate skeleton of the 12th nucleic acid from the 5′ end was substituted with a phosphothioate bond.

Further, each reaction solution was prepared to have the composition listed in Table 2 and introduced into the wells of a fluid device provided with a well array. The well array had approximately 930000 wells and the volume per well was approximately 1683 fL.

Subsequently, the fluid device was set in an aluminum block constant temperature bath (model “DTU-Mini”, TIETEC Corporation), heated at 66° C. for 25 minutes, and observed with a microscope (product name “All-in-one Fluorescence Microscope”, model “BZ-X810”, Keyence Corporation).

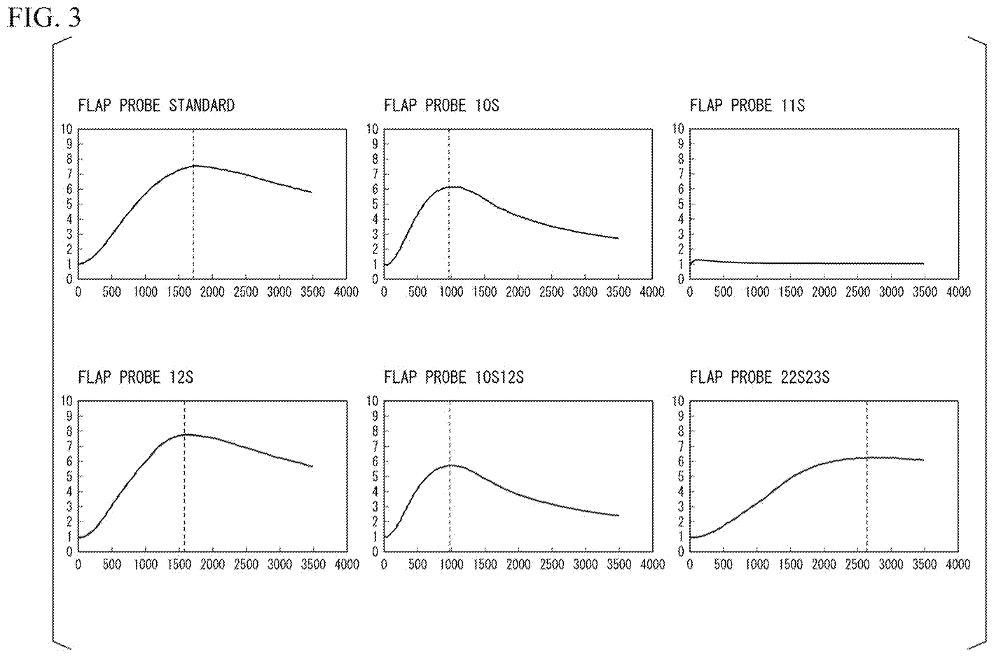

Subsequently, based on the image observed with the microscope, light emitting wells (S) and non-light emitting wells (N) were identified, and the brightnesses of the light emitting wells and the non-light emitting wells were calculated. Subsequently, the ratio between the brightness of the light emitting wells and the brightness of the non-light emitting wells, that is, the S/N ratio was calculated. FIG. 3 is a graph showing the S/N ratio in a case where each flap probe was used.

As shown in FIG. 3, it was confirmed that in a case where a phosphodiester bond in the sugar phosphate skeleton of at least any one nucleic acid at positions before and after one nucleotide of the 11th nucleic acid from the 5′ end which is the cleavage site in the flap probe, that is, at least any one nucleic acid of the 10th nucleic acid from the 5′ end or the 12th nucleic acid from the 5′ end was substituted with a phosphothioate bond, the time at which the S/N ratio was maximized was shortened by several hundred seconds as compared with a case where the flap probe standard, namely control, was used.

As shown in the above-described results, it has been suggested that the reaction speed of ICA can be increased by imparting the cleavage resistance to the flap endonuclease to at least any one nucleic acid at a position before one nucleotide and at a position after one nucleotide of the cleavage position due to the flap endonuclease.

### Example 2

(Examination on Concentration of Flap Probe)

ICA was performed by changing the concentration of the flap probe at which satisfactory results were obtained in Example 1, and the reactivity was compared.

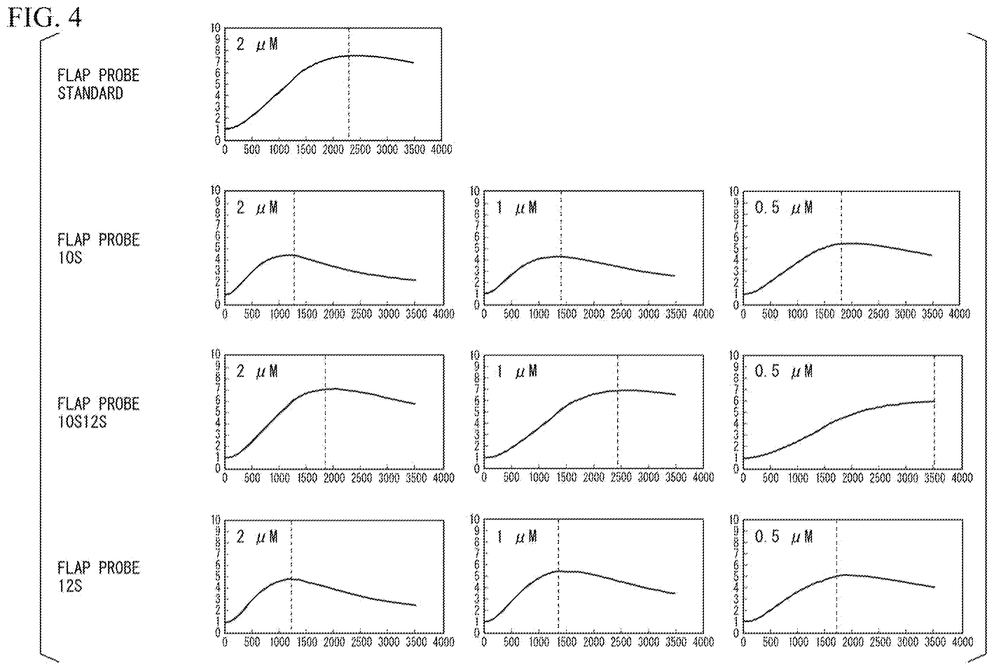

Specifically, the S/N ratios were calculated by the same method as that in Example 1 except that the concentrations of the flap probes 10S, 12S, and 10S12S in the reaction solution were changed to 0.5 μM, 1 μM, and 2 μM. Further, the S/N ratio was calculated in the same manner as described above even under a condition that the concentration of the flap probe standard in the reaction solution was set to 2 μM as a control. FIG. 4 is a graph showing the S/N ratio in a case where each of flap probes having different concentrations was used.

As shown in FIG. 4, it was confirmed that in a case where the amount of the flap probes 10S and 12S used was reduced to 0.5 μM, the time at which the S/N ratio was maximized was faster than in the case where the flap probe standard was used. It was confirmed that in a case where the amount of the flap probe 10S12S used was reduced to 1 μM, the time at which the S/N ratio was maximized was faster than in the case where the flap probe standard was used.

As shown in the above-described results, it has been found that in a case where the cleavage resistance to the flap endonuclease was imparted to at least any one nucleic acid at the position before one nucleotide or at the position after one nucleotide of the cleavage position due to the flap endonuclease so that the reaction speed of ICA was increased, the amount of the flap probe used was reduced.

According to the flap probe of the present embodiment, it is possible to provide a flap probe for ICA in which the cleavage position due to a flap endonuclease is controlled. According to the method of the present embodiment, it is possible to control the cleavage position of the flap probe due to the flap endonuclease in ICA. The kit for ICA according to the present embodiment includes the flap probe and can control the cleavage position of the flap probe due to the flap endonuclease in ICA. The method of detecting a target nucleic acid using ICA according to the present embodiment uses the flap probe, and the cleavage position of the flap probe due to the flap endonuclease can be controlled in ICA.

While preferred embodiments of the invention have been described and illustrated above, it should be understood that these are exemplary of the invention and are not to be considered as limiting. Additions, omissions, substitutions, and other modifications can be made without departing from the scope of the invention. Accordingly, the invention is not to be considered as being limited by the foregoing description and is only limited by the scope of the appended claims.

What is claimed is:

1. A method of controlling a cleavage position in an invasive cleavage assay, the method comprising:
bringing a target nucleic acid, a flap probe, and an invasive probe into contact with each other,
wherein the flap probe is a single-stranded oligonucleotide having a sequence complementary to a target nucleic acid, and
the flap probe has a structure in which at least any one position of a position before one nucleotide or a position after one nucleotide of the cleavage position due to the flap endonuclease has cleavage resistance to the flap endonuclease.

2. The method according to claim 1,
wherein the structure having cleavage resistance to the flap endonuclease is a phosphothioate bond.

3. A flap probe which is used in an invasive cleavage assay,
wherein the flap probe is a first single-stranded oligonucleotide having a first sequence complementary to a first position of a target nucleic acid, and
the flap probe has a structure in which at least any one position of a position before one nucleotide or a position after one nucleotide of a cleavage position due to a flap endonuclease has cleavage resistance to the flap endonuclease.

4. The flap probe according to claim 3,
wherein the structure having cleavage resistance to the flap endonuclease is a phosphothioate bond.

5. A kit for an invasive cleavage assay, comprising:
the flap probe according to claim 3;
an invasive probe;
a fluorescent probe; and
a flap endonuclease,
wherein the invasive probe is a second single-stranded oligonucleotide having a second sequence complementary to a second position of the target nucleic acid,
the first and second sequences and are different from each other,
the first and second positions are located adjacent to each other in the target nucleic acid,
the fluorescent probe is a third single-stranded oligonucleotide labeled with a fluorescent substance and a quenching substance,
a 5′ side of the fluorescent probe has a hairpin structure formed by self-hybridization, and
a 3′ side of the fluorescent probe has a sequence complementary to a nucleic acid fragment included in the flap probe.

6. A kit for an invasive cleavage assay, comprising:
the flap probe according to claim 4;
an invasive probe;
a fluorescent probe; and
a flap endonuclease,
wherein the invasive probe is a second single-stranded oligonucleotide having a second sequence complementary to the second position of the target nucleic acid,
the first sequence and a second sequence complementary are different from each other,
the first and second positions are located adjacent to each other in the target nucleic acid,
the fluorescent probe is a third single-stranded oligonucleotide labeled with a fluorescent substance and a quenching substance,
a 5′ side of the fluorescent probe has a hairpin structure formed by self-hybridization, and
a 3′ side of the fluorescent probe has a sequence complementary to a nucleic acid fragment included in the flap probe.

7. A method of detecting a target nucleic acid using an invasive cleavage assay, the method comprising:
using the flap probe according to claim 3.

8. A method of detecting a target nucleic acid using an invasive cleavage assay, the method comprising:
using the flap probe according to claim 4.


SEQUENCE LISTING (12 sequences)

SEQ ID NO: 1 - DNA, 105 bp, synthetic construct
  source 1..105: mol_type = other DNA
ccgaagggcatgagctgcgtgatgagctgcacggtggaggtgaggcagatgcccagcaggcggcacacgtgggggttgtccacgctggccatcacgtaggcttcc

SEQ ID NO: 2 - DNA, 24 bp, synthetic construct
  source 1..24: mol_type = other DNA
  misc_binding 12..24: bound_moiety = Binds to the 6th to 19th base sequence from the 5' end of the target nucleic acid
cgcgccgaggcgcagctcatgccc

SEQ ID NO: 3 - DNA, 24 bp, synthetic construct
  source 1..24: mol_type = other DNA
  modified_base 10^11: mod_base = OTHER; note = Phosphodiester bond is replaced with phosphorothioate bond
  misc_binding 12..24: bound_moiety = Binds to the 6th to 19th base sequence from the 5' end of the target nucleic acid
cgcgccgaggcgcagctcatgccc

SEQ ID NO: 4 - DNA, 24 bp, synthetic construct
  source 1..24: mol_type = other DNA
  modified_base 11^12: mod_base = OTHER; note = Phosphodiester bond is replaced with phosphorothioate bond
  misc_binding 12..24: bound_moiety = Binds to the 6th to 19th base sequence from the 5' end of the target nucleic acid
cgcgccgaggcgcagctcatgccc

SEQ ID NO: 5 - DNA, 24 bp, synthetic construct
  source 1..24: mol_type = other DNA
  modified_base 12^13: mod_base = OTHER; note = Phosphodiester bond is replaced with phosphorothioate bond
  misc_binding 12..24: bound_moiety = Binds to the 6th to 19th base sequence from the 5' end of the target nucleic acid
cgcgccgaggcgcagctcatgccc

SEQ ID NO: 6 - DNA, 24 bp, synthetic construct
  source 1..24: mol_type = other DNA
  modified_base 10^11: mod_base = OTHER; note = note= Phosphodiester bond is replaced with phosphorothioate bond.
  modified_base 12^13: mod_base = OTHER; note = Phosphodiester bond is replaced with phosphorothioate bond
  misc_binding 12..24: bound_moiety = Binds to the 6th to 19th base sequence from the 5' end of the target nucleic acid
cgcgccgaggcgcagctcatgccc

SEQ ID NO: 7 - DNA, 24 bp, synthetic construct
  source 1..24: mol_type = other DNA
  modified_base 22^23: mod_base = OTHER; note = note= Phosphodiester bond is replaced with phosphorothioate bond.
  modified_base 23^24: mod_base = OTHER; note = note= Phosphodiester bond is replaced with phosphorothioate bond.
  misc_binding 12..24: bound_moiety = Binds to the 6th to 19th base sequence from the 5' end of the target nucleic acid
cgcgccgaggcgcagctcatgccc

SEQ ID NO: 8 - DNA, 19 bp, synthetic construct
  source 1..19: mol_type = other DNA
  misc_binding 1..18: bound_moiety = Binds to the 20th to 37th base sequence from the 5' end of the target nucleic acid
ccaccgtgcagctcatcaa

SEQ ID NO: 9 - DNA, 11 bp, synthetic construct
  source 1..11: mol_type = other DNA
  misc_binding 1..10: bound_moiety = Bind to the 25th to 34th base sequence from 5' end of fluorescent probe
cgcgccgaggc

SEQ ID NO: 10 - DNA, 10 bp, synthetic construct
  source 1..10: mol_type = other DNA
  misc_binding 1..10: bound_moiety = Bind to the 25th to 34th base sequence from 5' end of fluorescent probe
cgcgccgagg

SEQ ID NO: 11 - DNA, 12 bp, synthetic construct
  source 1..12: mol_type = other DNA
  misc_binding 1..10: bound_moiety = Bind to the 25th to 34th base sequence from 5' end of fluorescent probe
cgcgccgaggcg

SEQ ID NO: 12 - DNA, 34 bp, synthetic construct
  source 1..34: mol_type = other DNA
  modified_base 1: mod_base = OTHER; note = Alexa Fluor (registered trademark) 488 is bound to the base.
  modified_base 5: mod_base = OTHER; note = BHQ (registered trademark)-1 is bound to a base.
tcttagccggttttccggctgagacctcggcgcg